(19) United States
(12) Plant Patent Application Publication
(10) Pub. No.: US 2017/0127591 P1
(43) Pub. Date: May 4, 2017

(54) Hop plant named 'HBC 520'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 15/330,752

(22) Filed: Nov. 1, 2016

Related U.S. Application Data
(60) Provisional application No. 62/285,568, filed on Nov. 2, 2015.

(51) Int. Cl. A01H 5/02

(52) U.S. Cl. PLT/236

(57) ABSTRACT

A new hop plant named ‘HBC 520’ is disclosed. ‘HBC 520’ is used for its exceptional and unique aromatic qualities.

### CROSS-REFERENCE TO RELATED APPLICATIONS

This application claims priority of co-pending provisional application Ser. No. 62/285,568 as filed on Nov. 2, 2015.

### STATEMENT REGARDING FEDERALLY SPONSORED RESEARCH OR DEVELOPMENT

None

Genus and species: Humulus lupulus.

Variety denomination: ‘HBC 520’.

### BACKGROUND OF THE INVENTION

‘HBC 520’ is a product of a controlled breeding program carried out by the inventors in the Yakima Valley of Washington State. ‘HBC 520’ was one of several seedlings resulting from controlled cross made in 2006 with female parent HBC 182 (unpatented) and male parent 986-2 (unpatented). A single plant of ‘HBC 520’ was selected in 2009, and in 2010 was expanded to 30 plants, which were planted in the area of Toppenish, Wash. The plants were observed and evaluated for several years, and in 2014 a 1 acre test plot of ‘HBC 520’ was established in the area of Moxee, Wash. Throughout several generations of asexual propagation, ‘HBC 520’ has been observed to retain its distinctive characteristics and remain true to type.

### BRIEF DESCRIPTION OF THE PHOTOGRAPHS

FIG. 1 illustrates a mature ‘HBC 520’ hop plant grown on a trellis;

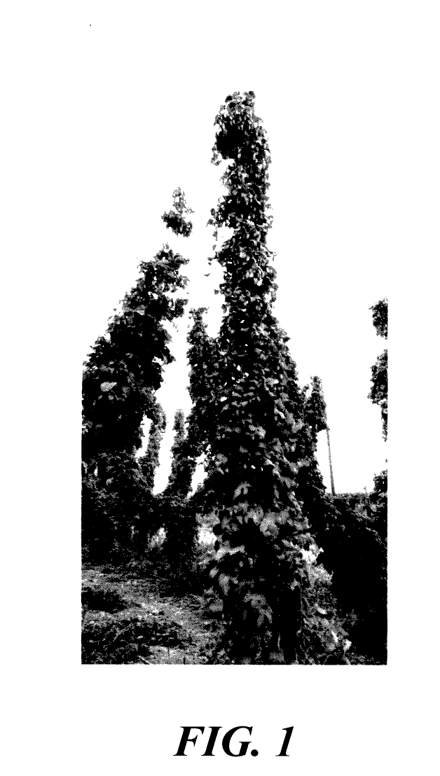

FIG. 2 illustrates whole cones and cross sections of cones of the ‘HBC 520’ hop plant;

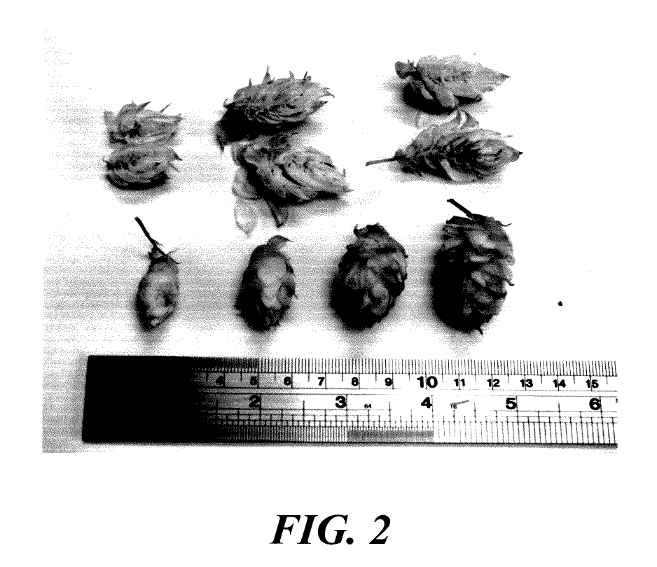

FIG. 3 illustrates leaves from a mature ‘HBC 520’ hop plant.

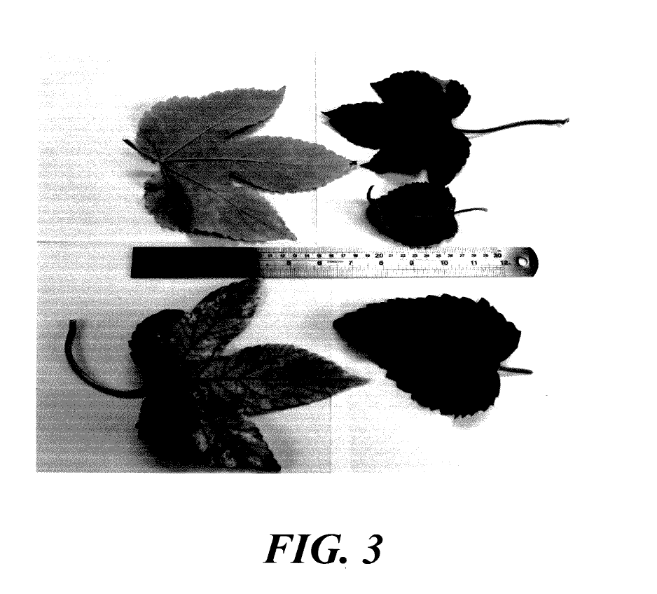

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2009-2015 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid (2n=2x).
Sex: Female.
Use: Brewing beer and ale.
Harvest date: September (during 2009 to 2015 growing seasons at Toppenish, Wash.).
Quality Characteristics (Averages):
   Alpha acid (as % of cone weight).—11.3%.
   Beta acid (as % of cone weight).—5.3%.
   Cohumulone (as % of alpha acids).—22%.
   Storageability (% loss).—12%.
Oil analytical characteristics (averages):
   Total oil (ml/100 g of cones).—2.00% Oil.
   A-Pinene.—0.09% Total Oil.
   B-Pinene.—0.59% Total Oil.
   Myrcene.—45.00% Total Oil.
   2-methyl-butyl isobutyrate.—0.66% Total Oil.
   Limonene.—0.21% Total Oil.
   Linalool0.23% Total Oil.
   Caryophyllene.—20.91% Total Oil.
   Farnesene.—0.09% Total Oil.
   Humulene.—16.04% Total Oil.
   Citral.—1.69% Total Oil.
   Geranyl acetate.—0.09% Total Oil.
   Citronellol.—1.39% Total Oil.
   Nerol.—0.14% Total Oil.
   Geraniol.—0.03% Total Oil.
   B-lonene.—0.01% Total Oil.
Bine:
   Color.—Yellow-Green 144D.
   Stripe present.—No.
   Stripe color.—Yellow-Orange 15A.
   Stipule direction.—Horizontal.
   Bine diameter.—10 mm at base; 11 mm at nine feet; 10 mm at terminal end of eighteen feet.
Leaf:
   Arrangement.—Opposite.
   Shape.—Palmate.
   Average length of mature leaf.—16.5 cm.
   Average width of mature leaf.—17.5 cm.
   Color of mature leaf upper surface.—Green 137A.
   Color of mature leaf lower surface.—Green 137A.
   Color of immature leaf upper surface.—Green 137B.
   Color of immature leaf lower surface.—Green 137A.
   Number of lobes.—5.
   Margin.—Serrate.
   Serrations per inch.—5.
   Average petiole length (mature).—65 mm.
   Petiole color at base.—Yellow-Green 144A.
   Venation.—Palmate.
   Vein color.—Yellow-Green 145B.
Cone:
   Avg. Length.—40 mm.
   Avg. diameter.—20 mm.
   Bract tip color.—Yellow-Green 144B.
   Bract base color.—Yellow-Green 145C.
   Bracteole color.—Yellow-Green 145C.
   Cone shape.—Oblong.
   Bract shape.—Orbicular.
   Bract tip shape.—Oval.
   Bracteole shape.—Ovate.
   Lupulin glands color.—Yellow-Orange 17C.

1. What is claimed is a new and distinct hop plant as shown and described herein.
